(19) United States
(12) Patent Application Publication
(10) Pub. No.: US 2007/0020346 A1
(43) Pub. Date: Jan. 25, 2007

(54) Botanical anticancer formulations

(76) Inventors: Hongtao Xing, New Territories (HK); Edgar Shiu Lam Liu, New Territories (HK); Sharon Luk, Ma On Shan (HK); Shiu Fun Pang, Kowloon (HK)

Correspondence Address: FISH & RICHARDSON PC, P.O. BOX 1022, MINNEAPOLIS, MN, 55440-1022, US

(21) Appl. No.: 11/417,888

(22) Filed: May 3, 2006

Related U.S. Application Data
(60) Provisional application No. 60/677,055, filed on May 3, 2005.

(51) Int. Cl. A61K 36/896; A61K 36/48

(52) U.S. Cl. 424/725; 424/753; 424/757

(57) ABSTRACT

This invention relates to a method of preparing a Schizandra, Trichosanthes, Glycine, or Yucca extract. Also disclosed are compositions containing at least two of these extracts, and methods of using the compositions for inducing apoptosis or cell cycle arrest and inhibiting angiogenesis or tumor cell metastasis.

### CROSS REFERENCE TO RELATED APPLICATION

Under 35 U.S.C. §119, this application claims priority to U.S. Provisional Application Ser. No. 60/677,055, filed May 3, 2005, the contents of which are incorporated herein by reference.

### BACKGROUND

Natural botanical products have a long history in medical applications. They are generally mild and have few side effects. By contrast, chemotherapy, routinely used to treat cancer, often causes nausea, vomiting, stomatitis, esophagitis, or diarrhea. Thus, there is a need to develop botanical anticancer formulations.

### SUMMARY

In one aspect, this invention features methods of preparing a Schizandra (e.g. Schizandra chinensis), Trichosanthes (e.g. Trichosanthes kirilowii maxim), Glycine (e.g. Glycine max—(L.)Merr.), or Yucca (e.g. Yucca schidigera) extract. The methods include first incubating a mixture containing [i] a part of Schizandra (e.g., a fruit), a part of Trichosanthes (e.g., a fruit), a part of Glycine, (e.g., a fruit), or a part of Yucca (e.g., a trunk) and [ii] an extracting solvent at an elevated temperature (e.g., 55° C. to 65° C.) for an extended period of time (e.g., 0.5 to 24 hours) to obtain an incubated mixture containing an insoluble material.

Preferably, a part of Schizandra or Trichosanthes and an extracting solvent are mixed at aria of 1:1 wt./vol. to 1:10 wt./vol. (e.g., 1:2 wt./vol. to 1:5 wt./vol.), and a part of Glycine or Yucca and an extracting solvent are mixed at a ratio of 1:1 wt./vol. to 1:20 wt./vol. (e.g., 1:5 wt./vol. to 1:10 wt./vol.). The extracting solvent can be water or a polar organic solvent. It can also be a mixture thereof (e.g., a 30-90% aqueous ethanol solution). An incubated mixture thus obtained can be optionally adjusted to a pH value between 4 and 10 (e.g., between 6 and 8).

After the incubation, the insoluble material in the mixture, whether adjusted or not, can be removed to afford a crude extract. The crude extract thus obtained from a part of Schizandra or Trichosanthes is placed in a matrix, such as a separation medium contained in a solid phase extraction column (e.g., a C18 reverse phase column) or a binding resin, and an eluting solvent (e.g., ethanol) is then passed through the matrix to give an eluent to be collected as a Schizandra or Trichosanthes extract for use, e.g., preparing a composition of this invention (see below). By contrast, the crude extract thus obtained from a part of Glycine or Yucca can be used with or without this process.

In another aspect, this invention features a composition containing at least two extracts selected from the group consisting of a Schizandra extract, a Trichosanthes extract, a Glycine extract, and a Yucca extract. For example, the compositions can contain all four extracts mixed at a ratio of 1-10:1-10:1-10:1-10 (e.g., 1:1:1:1, 1:2:3:4, 7:8:9:5, or 10:1:5:6). The Schizandra extract, the Trichosanthes extract, the Glycine extract, and the Yucca extract can be prepared according to the method described above.

In still another aspect, this invention features a method of inducing apoptosis or cell cycle arrest by contacting cells with the composition described above.

In a further aspect, this invention features a method of inhibiting angiogenesis or tumor cell metastasis, or treating a cell proliferation disorder. The method includes administering to a subject in need thereof an effective amount of the composition described above.

This invention also features the above-described composition for use in inducing apoptosis or cell cycle arrest, inhibiting angiogenesis or tumor cell metastasis, or treating a cell proliferation disorder, and the use of the composition for the manufacture of medicaments for carrying out these purposes.

The details of one or more embodiments of the invention are set forth in the description below. Other features, objects, and advantages of the invention will be apparent from the description and from the claims.

### DETAILED DESCRIPTION

This invention relates to a method of preparing a Trichosanthes, Schizandra, Glycine, or Yucca extract. Also within the scope of this invention are compositions containing at least two of these extracts and methods of using such compositions for inducing apoptosis or cell cycle arrest, inhibiting angiogenesis or tumor cell metastasis, or treating a cell proliferation disorder.

An extract of Trichosanthes, Schizandra, Glycine, or Yucca can be obtained from a part of each herb. The part can be a leaf, fruit, stem, root, or trunk. Schizandra, also known as magnolia vine and fruit of five flavors, belongs to the Schizandraceae family. It is a creeping vine with numerous clusters of tiny and bright red berries. Schizandraceae is native to northern China. Trichosanthes, also called gourd, belongs to the Cucurbitaceae family. It is mainly found in tropical and subtropical regions in Asia. Glycine, with its seeds known as soybean (also called soya, soja, or shoyu), belongs to the Fabaceae family. It is also native to tropical and warm temperate regions in Asia. Yucca belongs to the Agavaceae family. It is a desert tree that grows ubiquitously in Mexico and the United States. These herbs are commercially available, e.g., MJ Puehse & Company, El Dorado Hills, Calif.

To prepare an extract, a part of Trichosanthes, Schizandra, Glycine, or Yucca can first be physically disintegrated (e.g., sliced) and then dried. It can be optionally fermented before extraction. A part of Trichosanthes, Schizandra, Glycine, or Yucca thus obtained is incubated with an extracting solvent at an elevated temperature. The extracting solvent can be water, a polar organic solvent, or a mixture thereof at any suitable ratio. The term “polar organic solvent” refers to any organic solvent that contains a polar molecule and is generally miscible with water. Examples include, but are not limited to, ethanol, acetonitrile, and mixtures of these solvents.

A part of Schizandra or Trichosanthes and an extracting solvent can be mixed at a ratio of 1:1 wt./vol. to 1:10 wt./vol. (e.g., 1:2 wt./vol. to 1:5 wt./vol.). Apart of Glycine or Yucca and an extracting solvent can be mixed at a ratio of 1:1 wt./vol. to 1:20 wt./vol. (e.g., 1:5 wt./vol. to 1:10 wt./vol.). The mixture can be incubated in an ultrasonicator (for small scale production) or an extraction container (for large scale production) at an elevated temperature (e.g., 55° C. to 65° C. or 58° C. to 62° C.) for 0.5 to 48 hours (e.g., 1 to 24 hours or 3 to 5 hours) with sonication/stirring to obtain an incubated mixture.

The incubated mixture can be optionally adjusted to pH 4-10 (e.g., pH 6.5-7.5) by adding an alkaline substance during or after the incubation. For example, an alkaline substance can be added to the incubation mixture after incubation for a period of time. The incubation can continue for an extended period of time after the pH adjustment. Examples of a suitable alkaline substance include, but are not limited to, sodium hydroxide, sodium carbonate, and sodium bicarbonate.

Upon completion of the incubation, the insoluble material in the incubated mixture (with or without a pH adjustment) is removed by a suitable method (e.g., decantation, filtration, or centrifugation) to afford a crude extract. In small scale production, the mixture can be filtered through a cheese cloth or medical gauze to obtain a filtrate. Any remaining insoluble material in the filtrate can be further removed by centrifugation. In large scale production, the mixture can be filtered through a metal mesh filter (e.g., 100-400 mesh).

When a mixture of water and an polar organic solvent (e.g., ethanol) is used as an extracting solvent to incubate an herb, the crude extract thus obtained can be optionally concentrated by removing the organic solvent (e.g., using a rotary evaporator). The crude extract can be subjected to two liquid-liquid extractions. It can first be extracted with a non-polar organic solvent (e.g. n-hexane) to remove any contaminants (e.g., pigments, lipids, fatty acids, or waxes). It can be further extracted with a polar organic solvent (e.g. ethyl acetate, methylenechloride, or chloroform) to transfer desired ingredients into the organic solvent and obtain an crude extract in an organic solvent.

The crude extract from a part of Schizandra or Trichosanthes (with or without liquid-liquid extractions) is further subjected to a solid phase extraction, i.e., by first loading the extract onto a matrix and then eluting the matrix with a solvent. In small scale production, the matrix can be a separation medium contained in a C18 reverse phase column, normal phase column, ion-exchange column, or size-exclusion column. In large scale production, the matrix can be a binding resin (e.g. D101, D1300, X-5, AB-8, H103, D204, or DM11). In either case, an eluting solvent is then passed through the matrix to give an eluent. Examples of a suitable eluting solvent include ethanol, methanol, isopropanol, water, acetonitrile, and a mixture thereof. The eluent is collected as a Schizandra or Trichosanthes extract, which can be used to prepare a composition of this invention. By contrast, the crude extract from a part of Glycine or Yucca (with or without liquid-liquid extractions) can be used without undergoing the just-mentioned process.

Other methods that can be used to purify the crude extract obtained above include, but are not limited to, thin layer chromatography, gas chromatography, liquid chromatography, and high-performance liquid chromatography.

A skilled technician will appreciate that solvents, separation methods, and elution methods not explicitly recited in the foregoing may be successfully utilized in the practice of the present invention, and that these alternate materials and methods may be determined without undue experimentation.

Two or more of the Schizandra, Trichosanthes, Glycine, and Yucca extracts obtained above can be mixed together in a suitable ratio to obtain a mixture, i.e., a composition of this invention. For example, the four extracts can be mixed together at a ratio of 1:1:1:1 (dry weight). If desired, the solvent in the mixture can be removed, e.g., by lyophilization or spray drying.

This invention thus also covers contacting cells with a composition of this invention to induce apoptosis or cell cycle arrest. It also covers administering to a subject an effective amount of the composition to inhibit angiogenesis or tumor cell metastasis or to treat a cell proliferation disorder. “An effective amount” refers to the amount that is required to confer a therapeutic effect on the treated subject. Effective doses will vary, as recognized by those skilled in the art, depending on the types of diseases treated, route of administration, excipient usage, and the possibility of co-usage with other therapeutic treatment. A composition can be administered orally or topically.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

The compositions described above can be preliminarily screened for inducing apoptosis or cell cycle arrest, inhibiting angiogenesis or tumor cell metastasis, or treating a cell proliferation disorder by in vitro assays (such as those described in Examples 3-7 below) and then confirmed by animal experiments (such as that described in Example 8 below) and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent.

### EXAMPLE 1

### Small Scale Production of a Four-Extract Mixture from Schizandra, Trichosanthes, Glycine and Yucca

900 g of Schizandra fruits were oven dried and grinded into powder. 40% ethanol was added into the Schizandra powder in a 10 L bottle (˜1 kg herb:˜4 L ethanol). The mixture was incubated in a 60° C. ultrasonicator for an hour. Sodium carbonate was added to adjust the mixture to approximately pH 7. The mixture was incubated in a 60° C. ultrasonicator overnight with occasional sonication. The insoluble substance in the mixture was removed by passing the mixture through a cheese cloth. The sedimentation was then spun down and a clear filtrate was collected. The filtrate was further purified by a solid phase extraction method using a C18 reverse phase column. After the C18 reverse phase column was eluted with ethanol, the eluent was collected in sample collection tubes to obtain a Schizandra extract.

10 kg of Trichosanthes fruits were crushed into small pieces and oven dried. 40% ethanol was added into the trichosanthes in a 50 L container (˜1 kg herb:˜3 L ethanol). The mixture was incubated in a 60° C ultrasonicator overnight with occasional sonication. Sodium carbonate was then added to adjust the mixture to approximately pH 7. The insoluble substance in the mixture was removed by passing the mixture through a cheese cloth. The sedimentation was spun down and a clear filtrate was collected. The filtrate was further purified by a solid phase extraction method using a C18 reverse phase column. After the C18 reverse phase column was eluted with ethanol, the eluent was collected in sample collection tubes to obtain a Trichosanthes extract.

32 g of Glycine were oven dried and grinded into powder. 40% ethanol was added into the glycine powder in a 1 L bottle (˜1 g herb:˜10 ml ethanol). The mixture was incubated in a 60° C. ultrasonicator overnight with occasional sonication. The insoluble substance in the mixture was removed by passing the mixture through a cheese cloth. The sedimentation was spun down and a clear filtrate was collected to obtain a glycine extract.

32 g of Yucca trunks were oven dried and grinded into powder. 40% ethanol was added into the Yucca powder in a 1 L bottle (˜1 g herb:˜10 ml ethanol). The mixture was incubated in a 60° C. ultrasonicator overnight with occasional sonication. Sodium carbonate was then added to adjust the mixture to approximately pH 7. The insoluble substance in the mixture was removed by passing the mixture through a cheese cloth. The sedimentation was then spun down and a clear filtrate was collected to obtain a Yucca extract.

After the extract preparation, the dry weights of the four extracts were determined (Schizandra 18 g, Trichosanthes 20 g, Glycine 15 g, and Yucca 26 g). The four extracts were mixed together in a ratio of 1:1:1:1 (dry weight). The final mixture was then evaporated using a rotary evaporator. The concentrate was frozen and lyophilized to give a four-extract mixture (1:1:1:1).

### EXAMPLE 2

### Large Scale Production of a Four-Extract Mixture from Schizandra, Trichosanthes, Glycine, and Yucca

8.6 kg of Schizandra fruits were oven dried. 40% ethanol was added into the Schizandra powder (˜1 kg herb:˜4 L ethanol) in an extraction container. The extraction temperature was maintained at around 60° C. with constant stirring for 24 hours. Sodium carbonate was added to adjust the mixture to approximately pH 6.5-7.5 after the extraction process. The insoluble substance in the mixture was removed by passing the mixture through a metal mesh (<100 mesh). The solution was further passed through another mesh filter (100-400 mesh) and a clear filtrate was collected. The clear filtrate was then passed through a C18 reverse phase column. After the column was eluted with ethanol, the eluent was collected to obtain a Schizandra extract.

99 kg of Trichosanthes fruits were crushed into small pieces and oven dried. 40% ethanol was added into the trichosanthes in an extraction container (˜1 kg herb:˜3 L ethanol). The extraction temperature was maintained at around 60° C. with constant stirring for 24 hours. Sodium carbonate was added to adjust the mixture to approximately pH 6.5-7.5 after the extraction process. The insoluble substance in the mixture was removed by passing the mixture through a metal mesh (<100 mesh). The solution was further passed through another mesh filter (100-400 mesh) and a clear filtrate was collected. The filtrate was then passed through a C18 reverse phase column. After the column was eluted with ethanol, the eluent was collected to obtain a Trichosanthes extract.

300 g of Glycine were oven dried and grinded into powder. 40% ethanol was added into the Glycine powder in an extraction container (˜1 g herb:˜10 ml ethanol). The extraction temperature was maintained at around 60° C. with constant stirring for 24 hours. The insoluble substance in the mixture was removed by passing the mixture through a metal mesh (<100 mesh). The solution was further passed through another mesh filter (100-400 mesh) and the filtrate was collected to obtain a Glycine extract

250 g of Yucca trunks were oven dried and grinded into powder. 40% ethanol was added into the Yucca powder in an extraction container (˜1 g herb:˜10 ml ethanol). The extraction temperature was maintained at around 60° C. with constant stirring for 24 hours. The insoluble substance in the mixture was removed by passing the mixture through a metal mesh(<100 mesh). The solution was further passed through another mesh filter (100-400 mesh) and the filtrate was collected to obtain a Yucca extract.

The dry weights of the four extracts were determined (Schizandra, 170 g, Trichosanthes 200 g, Glycine 140 g, and Yucca 210 g). The four extracts were mixed together at a ratio of 1:1:1:1 (dry weight). The mixture was concentrated to around 10 L. The concentrate was then spray dried to give a four-extract mixture (1:1:1:1).

### EXAMPLE 3

### Schizandra, Trichosanthes, Yucca, and Glycine Extract Mixtures Produced Cytotoxic Effect on Human Cancer Cell Lines

61 human leukemia, melanoma, colon, breast, prostate, lung, gastric, liver, CNS, ovary, and kidney cell lines were used to determine the cytotoxic effect of Schizandra, Trichosanthes, Yucca, and Glycine extract mixtures. The cell lines were cultured in their appropriate medium supplemented with fetal bovine serum and 1% Penicillin/Streptomycin at 5% CO₂, 37° C.

Sulforhodamine B (SRB) assay was applied to determine the cytostatic effect of the extracts combinations. SRB is a dye that binds to cellular proteins and is soluble in a base. The biomass of total protein is measured at 520 nm by using a plate reader.

Cells were inoculated into 96-well microtiter plates including “Time zero” (Tz) plates in 100 μl at cell concentrations from 5,000 to 40,000 cells per well. The cells were incubated at 37° C. for 24 hours. The extracts combinations were dissolved in dimethyl sulfoxide and then added to the cells of final concentrations ranging from 0.977 to 500 μg/ml for 48 hours at 37° C. with 5% CO₂. Cold Trichloroacetic acid (TCA) was added at final concentrations of 10% (w/v) to adherent cells and 16% (w/v) to suspension cells for cell fixation for at least 60 minutes at 4° C. The supernatant was discarded and the plates were washed in tap water for 5 times and air dried. SRB solution (0.4% (w/v)) was added to stain the cells for 10 minutes at room temperature. The plates were then washed with 1% acetic acid 3 times and air dried. Bound SRB was solubilized with 100-200 μl per well of 10 mM Trizma base and absorbance was measured at a wavelength of 520 nm.

The percentage of growth inhibition was calculated as follows:
% of growth inhibition=100−{[(Ti−Tz)/(C−Tz)]×100}

Where: Ti=Corrected absorbance of treatment well
Tz=Corrected absorbance of time zero well
C=Corrected absorbance of control well

The growth inhibition of 50% (GI₅₀) was obtained from the dose response curve of percentage of inhibition against dosage using Prism Software.

Different Schizandra, Trichosanthes, Yucca, and Glycine extract mixtures inhibited the growth of 61 cancer cell lines differently. For the Trichosanthes extract, the GI₅₀ values varied from 2.38 to 60.23 μg/ml, which were much lower than those for the Schizandra, Glycine, and Yucca extracts. Trichosanthes and Yucca extract mixtures at ratios 2:1 and 2:3 produced the corresponding GI₅₀ values of 17.7 and 12.7 μg/ml in hepatic cancer cells, respectively. Moreover, Trichosanthes and Yucca extract mixtures at ratios 1:3 and 1:9 produced corresponding GI₅₀ values of 13.5 and 20 μg/ml in gastric cancer cells, respectively. Schizandra, Trichosanthes and Yucca extract mixtures, at ratios of 9:4:6 and 35:7:34, inhibited hepatic caner cell growth, with the GI₅₀ values being 24.3 and 67.3 μg/ml, respectively. In addition, Schizandra, Trichosanthes and Yucca extract mixtures, at ratios of 9:1:3 and 35:27:15, inhibited gastric cancer cell growth, with the GI₅₀ values being 39.6 and 29.1 μg/ml, respectively. The four-extract mixture (1:1:1:1) inhibited cancer cell proliferation in a dose-dependent manner. The GI₅₀ values of this mixture on the 61 cell lines ranged from 4.4 to 259.7 μg/ml.

### EXAMPLE 4

### A Four-Extract Mixture Induced Cytokine Secretion from THP-1, Jurkat and 28SC Cells

Monocyte cell lines (THP-1 and 28SC) and T-lymphocyte cell line (Jurkat) were purchased from American Type Culture Collection. These cells were cultured in their appropriate medium supplemented with fetal bovine serum (FBS) and 1% Penicillin/Streptomycin at 5% CO₂, 37° C.

TNF-α, IL-1β, and IL-8 secretion was measured in Jurkat cells (THP-1, IFN-γ, and IL-2) and in 28SC cells (IL-6). Cells (5×10⁵ cells/well) were incubated with a four-extract mixture (1:1:1:1) for 4 hours prior to cytokines measurement following the OptEIA ELISA SET manual (BD Pharmingen, San Diego, Calif., USA). Wells were coated with 100 μl per well of capture antibody, diluted in a coating buffer, and incubated overnight at 4° C. The solution was aspired and the wells were washed three times with a wash buffer. The plate was blocked with 200 μl per well of an assay diluent and incubated at room temperature for 1 hour. Cytokine standards were diluted with an assay diluent into appropriate concentrations. One hundred microliter of a standard or sample solution was added into each well. The solution in each well was mixed and incubated at room temperature for 2 hours. The wells were aspired and washed 5 times with a wash buffer. One hundred microliter of a prepared working detector were added into each well and incubated at room temperature for 1 hour. It was followed by aspiration, washing, addition of 100 μl of substrate reagent, and incubation for 30 minutes at room temperature in the dark. The reaction was stopped by the addition of 50 μl of a stop solution. Absorbance at 450 nm and 570 nm (background correction) was obtained within 30 minutes after stoppage of the reaction. A graph for the absorbance against standard concentration was plotted and the amount of protein in the sample was obtained based on the standard curve.

Treatment with the mixture for 4 hours induced cellular secretion of TNF-α, IL-1β, and IL-8. The increases of TNF-α, IL-1β, and IL-8 in THP-1 cells were dosage dependent. Optimal doses of the mixture for induction of different cytokines were different. The optimal doses for inducing the secretion of TNF-α, IL-1β, and IL-8 were 200 μg/ml, 50 μg/ml, and 100 μg/ml, respectively.

### EXAMPLE 5

### A Four-Extract Mixture Produced Cell Cycle Arrest on Human Cancer Cell Lines

Human leukemia cell lines (SUP-T1, HL60, THP-1, and Jurkat), colon cancer cell line (Caco2), breast cancer cell lines (MDA-MB-231 andMCF-7), prostate cancer cell lines (PC-3, DU145, and LNCap), lung cancer cell lines (A549, H1437, and H838), gastric cancer cell lines (AGS and NCI-N87), and liver cancer cell line (HepG2) were treated with a mixture.

Different types of cells, at a density of 5×10⁴ to 2×10⁵ cells/flask, were seeded in 25 cm² flasks. After 24 hours, or immediately for those suspension cells, the seeded cells were incubated with the mixture at a final concentration of below, at, or above the IC₅₀ value of that particular cell line for 48 hours. Cells were harvested, fixed in 1 ml 80% ethanol, and incubated at 4° C. for 15 minutes. After incubation, cells were centrifuged at 453 r.c.f. for 5 minutes and the cell pellets were resuspended in 500 μl propidium iodine (10 μg/ml) containing 300 μg/ml RNase. Cells were incubated on ice for 30 minutes and filtered with 53 μm nylon mesh. Cell cycle distribution was calculated from counting 10,000 cells with ModFit LT™ software (BD Biosciences, San Jose, Calif., USA) using FACScaliber (BD Biosciences, San Jose, Calif., USA).

The mixture exhibited a dose-dependent cell cycle arrest effect. After treatment with the mixture for 48 hours, cells in the G2/M population increased significantly, compared to the controls for all cancer cell lines except for AGS and LNCap (slight increase) -and Jurkat (no change). The effect of the mixture on the different cancer cells appeared to be dose-dependent. SUP-T1 leukemia cells were treated with the mixture at three different concentrations, i.e., 5, 10, and 20 μg/ml, for 48 hours. Aneuploidy was found at the concentrations 10 and 20 μg/ml. DU145 prostate cells were treated with the mixture for 48 hours at four different concentrations, i.e., 7, 14, 28, and 40 μg/ml. Aneuploidy was found at the concentration 7 μg/ml.

### EXAMPLE 6

### A Four-Extract Mixture Induced Apoptosis in Jurkat, THP-1 and DU145 Cell Lines

Jurkat, THP-1, and DU145 cells were seeded in a 6-well plate at a density of 1.0×10⁶ cells/well. After treatment with the mixture for 48 hours at 9 different concentrations, i.e., 2.5, 5, 7, 10, 14, 20, 28, 40, and 60 μg/ml, cells were trypsinized. The cells were then washed twice with PBS and 5×10⁵ cells were resuspended in a 500 ml binding buffer. 100 μl of cell suspension were transferred to a 5 ml culture tube and incubated with 10 μl of Annexin V antibodies and 10 μl of propidium iodine (10 μg/ml) containing 300 μg/ml RNase. The cells were gently vortexed and incubated for 15 minutes at room temperature in the dark. 400 μl of binding buffer were added to each tube and the cells were analyzed with a flow cytometer within 1 hour.

The mixture induced apoptosis in Jurkat, THP-1, and DU145 cells when compared with the control groups. Of note, it induced apoptosis in a dose-dependent manner.

### EXAMPLE 7

### A Four-extract Mixture Inhibited Breast Cancer Cell Metastasis

The effect of a four-extract mixture (1:1:1:1) to inhibit cancer cell adhesion and invasion was studied using breast cancer cell line MDA-MB-231 to adhere to an extracellular matrix (i.e., Matrigel, fibronectin, and laminin). Wells were coated with Matrigel (250 μg/cm²), fibronectin (10 μg/cm²), or laminin (5 μg/cm²). Cells (1×10⁵ per well) were resuspended into the wells in the presence of 3.9, 7.8, 15.6, 31.2, 62.5, 125, or 250 μg/ml of the mixture for 1-2 hours. After incubation, the medium in the well was discarded and 0.1% crystal violet was used to stain the cells that adhered to the bottom of the plate. Absorbance was read at 570 nm to determine cell adhesion in the presence of the mixture.

For the cell invasion assay, MDA-MB-231 (2.5×10⁴ cells/well) was added into an invasion chamber, which was coated with 250 μg/ml Matrigel. The assay mixtures were then incubated with the four-extract mixture for 24 hours at 7 different concentrations, i.e., 3.9, 7.8, 15.6, 31.2, 62.5, 125, and 250 μg/ml. After incubation, the cells that migrated through the chamber were trypsinized and stained with CyQuant GR Dye (Molecular Probes, Eugene, Oreg., USA). Fluorescence was measured at 480/520 nm to determine the cell invasion ratio.

The mixture, at dosages greater than 31.25 μg/ml, significantly inhibited the adhesion of the cancer cells to Matrigel, fibronectin, and laminin. Of note, it inhibited cell invasion in a dose-dependent manner.

### EXAMPLE 8

### A Four-extract Mixture Inhibited Prostate and Breast Cancer Xenograft Growth in Nude Mice

Human prostate carcinoma cell line PC-3 was used for xenograft implantation in male balb/c nu/nu mice (SIPPR/BK Laboratory Animal Ltd, Shanghai, China). A 100 μl solution containing 5×10⁶ cells was injected subcutaneously into the right flank of the nude mice. Seven days after the cell inoculation, a four-extract mixture (1:1:1:1), at 0.5, 1, or 2 mg/mouse per day, was given orally for a total of 35 days. The sizes of tumor were measured by a caliper once in 7-8 days. These sizes were compared with those of the group with vehicle treatment only.

Human breast cancer cell line MCF-7 was used for xenograft implantation in female balb/c nu/nu mice. Estradiol pellet (Innovative Research of America, Sarasota, Fla., USA) was inserted s.c. in the left flank of the nude mice. A week later, a 100 μl solution containing 5×10⁶ cells was injected subcutaneously into the right flank of the nude mice. Seven days after the cell inoculation, the mixture, at 0.5, 1, or 2 mg/mouse per day, was given orally for a total of 28 days. The tumor sizes were measured by a caliper once in 7-8 days. The sizes of tumor were compared with those of the vehicle treatment group.

The mixture, at 0.5, 1, and 2 mg/mouse per day, significantly inhibited prostate tumor growth in nude mice. At 2 mg/mouse per day, it significantly inhibited breast tumor growth.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A composition comprising at least two extracts selected from the group consisting of a Schizandra extract, a Trichosanthes extract, a Glycine extract, and a Yucca extract.

2. The composition of claim 1, wherein the composition contains a Schizandra extract, a Trichosanthes extract, a Glycine extract, and a Yucca extract.

3. The composition of claim 2, wherein the extracts are mixed at a ratio of 1-10:1-10:1-10:1-10.

4. The composition of claim 3, wherein the extracts are mixed at a ratio of 1:1:1:1.

5. A method of inducing apoptosis, comprising contacting cells with a composition of claim 1.

6. The method of claim 5, wherein the composition contains a Schizandra extract, a Trichosanthes extract, a Glycine extract, and a Yucca extract.

7. The method of claim 6, wherein the extracts are mixed at a ratio of 1-10:1-10:1-10:1-10.

8. The method of claim 7, wherein the extracts are mixed at a ratio of 1:1:1:1.

9. A method of inducing cell cycle arrest, comprising contacting cells with a composition of claim 1.

10. The method of claim 9, wherein the composition contains a Schizandra extract, a Trichosanthes extract, a Glycine extract, and a Yucca extract.

11. The method of claim 10, wherein the extracts are mixed at a ratio of 1-10:1-10:1-10:1-10.

12. The method of claim 11, wherein the extracts are mixed at a ratio of 1:1:1:1.

13. A method of inhibiting angiogenesis, comprising administering to a subject in need thereof an effective amount of a composition of claim 1.

14. The method of claim 13, wherein the composition contains a Schizandra extract, a Trichosanthes extract, a Glycine extract, and a Yucca extract.

15. The method of claim 14, wherein the extracts are mixed at a ratio of 1-10:1-10:1-10:1-10.

16. The method of claim 15, wherein the extracts are mixed at a ratio of 1:1:1:1.

17. A method of inhibiting tumor cell metastasis, comprising administering to a subject in need thereof an effective amount of a composition of claim 1.

18. The method of claim 17, wherein the composition contains a Schizandra extract, a Trichosanthes extract, a Glycine extract, and a Yucca extract.

19. The method of claim 18, wherein the extracts are mixed at a ratio of 1-10:1-10:1-10:1-10.

20. The method of claim 19, wherein the extracts are mixed at a ratio of 1:1:1:1.

21. A method of treating a cell proliferation disorder, comprising administering to a subject in need thereof an effective amount of a composition of claim 1.

22. The method of claim 21, wherein the composition contains a Schizandra extract, a Trichosanthes extract, a Glycine extract, and a Yucca extract.

23. The method of claim 22, wherein the extracts are mixed at a ratio of 1-10:1-10:1-10:1-10.

24. The method of claim 23, wherein the extracts are mixed at a ratio of 1:1:1:1.
